(19) United States
(12) Patent Application Publication
(10) Pub. No.: US 2016/0066849 A1
(43) Pub. Date: Mar. 10, 2016

(54) Activity Monitor and Rate-Adaptive Implantable Leadless Pacemaker

(71) Applicant: BIOTRONIK SE & Co. KG, Berlin (DE)

(72) Inventors: Marcelo Baru, Tualatin, OR (US); Warren Dabney, Lake Oswego, OR (US); J. Christopher Moulder, Portland, OR (US)

(21) Appl. No.: 14/818,653

(22) Filed: Aug. 5, 2015

Related U.S. Application Data
(60) Provisional application No. 62/045,568, filed on Sep. 4, 2014.

(51) Int. Cl. A61B 5/00; A61B 5/1473; A61B 5/145; A61N 1/365; A61N 1/375

(52) U.S. Cl. CPC A61B 5/686; A61N 1/36557; A61N 1/3756; A61B 5/14539; A61B 5/14735; A61B 5/4866; A61B 2562/125

(57) ABSTRACT

An implantable leadless pacemaker having a housing and two electrodes arranged on said housing. The two electrodes are made of biocompatible materials that have different sensitivities to pH changes. The two electrodes are connected to an activity monitoring unit that is adapted to determine an open-circuit potential difference between the two electrodes and to generate an activity signal derived from said open-circuit potential difference.

### CROSS-REFERENCE TO RELATED APPLICATIONS

This patent application claims the benefit of co-pending U.S. Provisional Patent Application No. 62/045,568, filed on Sep. 4, 2014, which is hereby incorporated by reference in its entirety.

### TECHNICAL FIELD

The present invention generally relates to an activity monitor and to a rate-adaptive implantable leadless pacemaker comprising an activity monitor.

### BACKGROUND

Leadless cardiac pacemakers are small devices implanted directly in a ventricle of a heart. Leadless cardiac pacemakers are preferably rate-adaptive to change pacing output dependent on patient activity. A problem is that sensing patient activity normally requires large circuitry with high power consumption for the application.

Leadless cardiac pacemakers disclosed in prior art adapt pacing rate based either on an activity sensor (accelerometer) or a temperature sensor, and associated circuitry, hermetically contained within the housing.

Leadless pacemaker design has strict size and power consumption requirements. A motion-sensing activity sensor (e.g., an accelerometer) contained within the housing to adapt the pacing rate occupies substantial space and its associated circuitry (for signal processing) requires a power consumption level that reduces the lifetime of the device. A temperature sensor-based pacemaker, on the other hand, has a slow response time, which is detrimental to useful pacing rate adaptation.

In view of the above, there is a need for an improved rate-adaptive implantable leadless pacemaker and activity monitor.

The present invention is directed toward overcoming one or more of the above-mentioned problems.

### SUMMARY

It is an object of the present invention to provide an improved leadless pacemaker and an activity monitor for monitoring metabolic demand.

According to the present invention, at least this object is achieved by an activity monitor for monitoring metabolic demand that has two electrodes that are made of biocompatible materials that have different sensitivities to pH changes. The two electrodes are connected to an activity monitoring unit that is adapted to determine an open-circuit potential difference between said two electrodes and to generate an activity signal derived from said open-circuit potential difference.

An object of the present invention is further achieved by an implantable leadless pacemaker having a housing and at least two electrodes arranged on said housing. The two electrodes are made of biocompatible materials that have different sensitivities to pH changes. The two electrodes are connected to an activity monitoring unit (i.e., an activity monitor) that is adapted and/or configured to determine an open-circuit potential difference between the two electrodes and to generate an activity signal derived from said open-circuit potential difference.

Thus, an object of the present invention is achieved by an activity monitor for monitoring metabolic demand and a leadless pacemaker with an activity monitor that utilizes the electrical properties of its electrodes in contact with blood to adapt the pacing rate.

Changes in metabolic needs, such as, for example, those caused by physical activities or emotional stress, will vary the venous blood pH within a narrow range around 7.35.

The activity monitoring unit of the activity monitor and the implantable leadless pacemaker of the present invention thus respond to both physical activity and emotional stress. The term “activity” is thus used in broad sense, because it includes both physical activity and emotional stress.

While pH-based rate-adaptive pacemakers using cardiac leads were proposed and tested in the late 1970s, they did not gain clinical acceptance as the measurement techniques proposed required an Ag/AgCl reference electrode, which is not biocompatible or suitable for long term implantation.

The activity monitor for monitoring metabolic demand and the leadless pacemaker of the present invention incorporate two electrodes made of biocompatible materials with different sensitivity to pH changes. A first material presents a Nernstian or super-Nernstian behavior with pH, i.e., its open-circuit potential varies with a large negative slope, for example, of about less than −50 mV/pH, and preferably less than −60 mV/pH, and the second material is either insensitive to pH or presents a smaller negative slope (sub-Nernstian behavior), e.g., a slope of more than −40 mV/pH, and preferably more than −30 mV/pH.

In the implantable pacemaker, the open-circuit potential difference between these two electrodes is periodically measured to allow adjusting the pacing rate. The materials of the present invention are also suitable for electrical stimulation, which allows implementing a rate-adaptive pacemaker with the minimum two electrodes required for pacing.

The technical advantages of this invention include, but are not limited to: 1) implementing a rate-adaptive leadless pacemaker re-utilizing features already required for pacemaker operation; 2) implementing a rate-adaptive leadless pacemaker with minimum power consumption required for rate adaptation; 3) reduced component count and size (no need for an accelerometer or temperature sensor) saves space; 4) fast response time compared to a temperature-based sensor; 5) rate adaptation using pH is also responsive to emotional stress, which cannot be achieved with an accelerometer or temperature-based activity sensor.

In a preferred embodiment of the implantable leadless pacemaker, the activity monitoring unit is adapted to periodically determine the open-circuit potential difference between said two electrodes. Preferably, the implantable leadless pacemaker further comprises a control unit (i.e., controller) that is connected to the activity monitoring unit (i.e., activity monitor) and that is adapted to adjust a pacing rate in response to said activity signal generated by said activity monitoring unit.

In preferred embodiments of the activity monitor or the implantable leadless pacemaker, the sub-Nernstian material is a coating made of or comprising poly(3,4-ethylenedioxythiophene):poly(styrenesulfonate).

In preferred embodiments of the activity monitor or the implantable leadless pacemaker, the super-Nernstian material is a coating made of or comprising iridium oxide (IrO).

Preferably, the super-Nernstian material is a coating made of or comprising a non-stoichiometric composition of iridium oxide, namely either IrO(2−x) or IrO(2+x), and in particular, IrO(1.5).

In preferred embodiments, the coating made of or comprising iridium oxide is deposited on the electrode surface using reactive physical vapor deposition (PVD).

A preferred embodiment of the implantable leadless pacemaker has a housing whereupon the two electrodes are arranged and wherein the activity monitoring unit is adapted to periodically determine the open-circuit potential difference between said two electrodes. The preferred implantable leadless pacemaker further comprises a control unit that is connected to the activity sensor and that is adapted to adjust a pacing rate in response to said activity signal generated by said activity monitoring unit.

Preferably, the activity monitoring unit or the control unit, or both, are configured to generate an activity signal indicating an increase of pacing rate or increasing the pacing rate, respectively, only when a sudden decrease of pH is determined.

It is further preferred when the activity monitoring unit or the control unit or both are configured to determine or to respond to the activity signal during diastole only.

Similarly, it is further preferred when the activity monitoring unit or the control unit, or both, are configured evaluate the activity signal so as to determine a contact with an inner heart wall and thus to monitor cardiac contraction during systole.

The implantable leadless pacemaker may further comprise impedance determination circuitry and a third electrode made of or comprising iridium oxide, which is connected to said impedance determination circuitry. In such embodiment, in which preferably the first and/or the second electrodes are also connected to the impedance circuitry, the impedance determination circuitry is configured to determine a tissue-electrode capacitance or changes thereof. The third electrode preferably has a smaller surface than the first and second electrode.

In preferred embodiments of both, the activity monitor for monitoring metabolic demand or the implantable leadless pacemaker itself are configured to perform long-term monitoring of pH.

### DESCRIPTION OF THE DRAWINGS

The above and other aspects, features, object, advantages and possible applications of the present invention will be more apparent from the following more particular description thereof presented in conjunction with the following drawings, and the appended claims, wherein:

FIG. 1 is an external view of a leadless implantable pacemaker;

FIG. 2 is a schematic block diagram of some internal components of the pacemaker of FIG. 1; and

FIG. 3 is a schematic block diagram of some internal components of an alternative embodiment of a pacemaker according to the present invention.

### DETAILED DESCRIPTION

The following description is of the best mode presently contemplated for carrying out the present invention. This description is not to be taken in a limiting sense, but is made merely for the purpose of describing the general principles of the present invention. The scope of the present invention should be determined with reference to the claims, which should be given their full breadth.

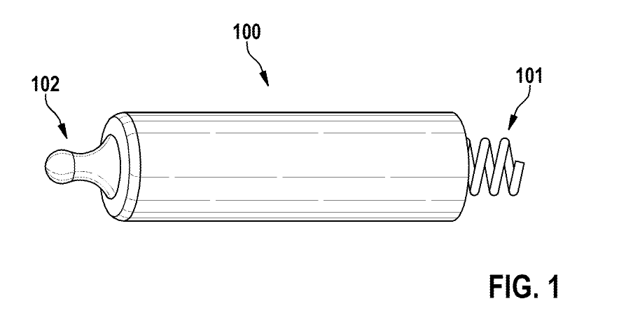

FIG. 1 shows an implantable leadless pacemaker with an elongate housing 100 and two electrodes 101 and 102. Each electrode is arranged at a respective longitudinal end of the housing 100.

Electrode 102 is a return electrode for pacing purposes. Electrode 101 is coated with poly(3,4-ethylenedioxythiophene):poly(styrenesulfonate) (also known as PEDOT:PSS), a biocompatible conductive polymer with a linear sub-Nernstian response in the pH range considered. Electrode 102, on the other hand, is coated with iridium oxide deposited with a state of oxidation that provides at least a linear Nernstian behavior with pH.

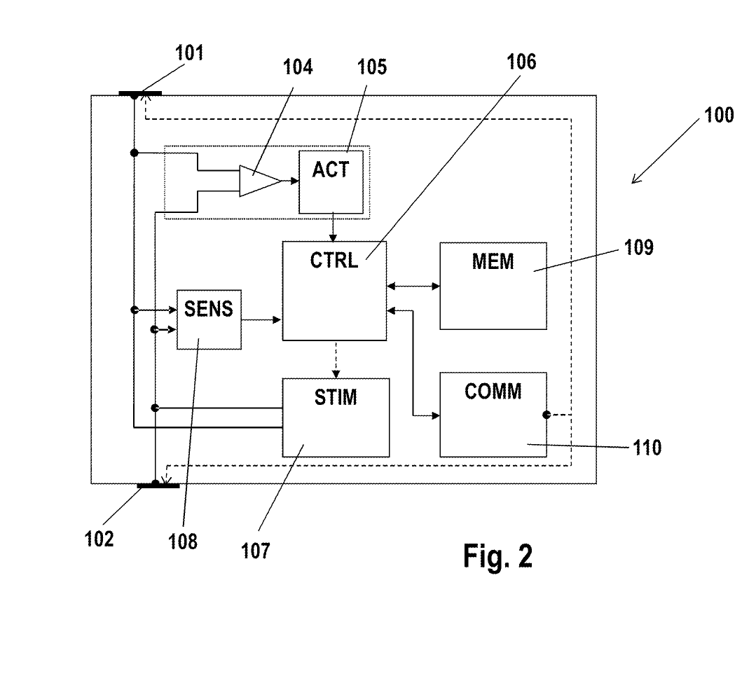

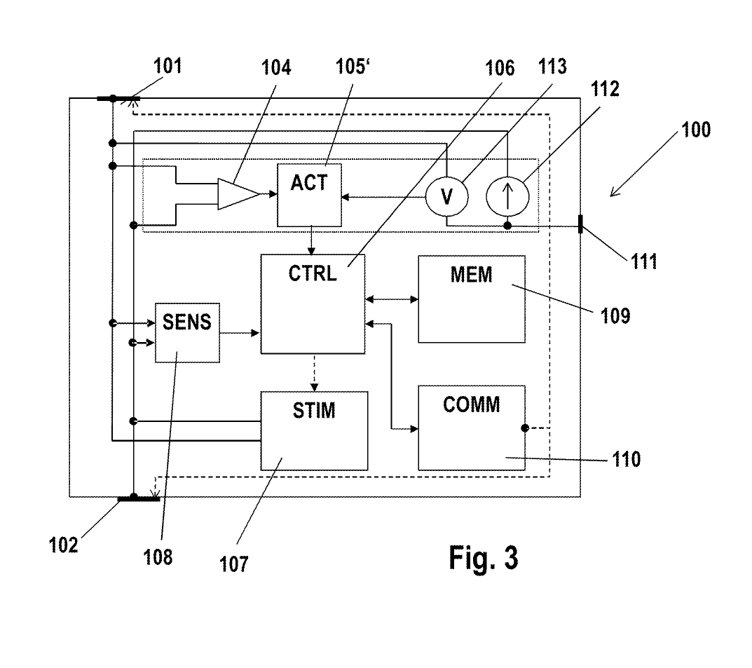

As shown in FIGS. 2-3, the electrodes 101 and 102 are connected to the inputs of an amplifier 104 contained within the housing 100 capable of measuring such varying open-circuit potentials difference. The measured potential is fed to an activity monitoring unit 105 (i.e., activity monitor) that generates an activity signal for a pacing control unit 106 (i.e., pacing controller). Pacing control unit 106 is connected to a stimulation unit 107 (i.e., stimulator) and a sensing unit 108 (i.e., sensor). Pacing control unit 106 is further connected to a memory 109 and a communication unit 110. By means of stimulation unit 107 and sensing unit 108, control unit 106 can apply demand pacing of a heart chamber through electrodes 101 and 102 in a manner known per se. By means of the activity signal provided by the activity monitoring unit 105, pacing can be rate-adaptive, that is, a respective pacing rate can be adapted to the metabolic demand of a respective person. In another embodiment, the sensing unit 108 is adapted to implement the tasks of 104 thus reducing the circuitry required for rate adaptation. Further, control unit 106 can interact with the electrodes 101 and 102 by means of the communication unit 110.

The implantable leadless pacemaker determines a metabolic demand by means of blood pH changes. Metabolic needs, including emotional stress, will vary the pH in the range of 7.35 to 7.29. Hence, with a resulting slope of −30 mV/pH, this translates into a full-range voltage variation of 1.8 mV. Sixteen steps of rate adaptation imply a resolution of approximately 112 μVN, which can be implemented with very low-power consumption.

The two electrodes 101 and 102 are made of biocompatible materials with different sensitivity to pH changes. A first material presents a Nernstian or super-Nernstian behavior with pH, i.e. its open-circuit potential varies with a large negative slope, for example of about −60 mV/pH, and the second material is either insensitive to pH or presents a smaller negative slope (sub-Nernstian behavior), e.g. a slope of −30 mV/pH.

Since pacing affects the ionic concentrations of the immediate layer next to an electrode, activity monitoring unit 105 and/or control unit 106 are configured to perform measurements for rate adaptation during diastole.

The control unit 106 is configured to increase the pacing rate only when sudden pH decreases are detected. This avoids runaway pacing rate changes that can occur from long-term pH changes in the body due to medication or other systemic effects.

In an alternative embodiment, electrodes 101 and 102 are coated with iridium oxide and PE-DOT:PSS, respectively.

FIG. 3 illustrates yet another embodiment, wherein the leadless pacemaker includes a third iridium oxide electrode 111 of much smaller area compared with the other iridium oxide electrode 102. pH variations will result in changes of the tissue-electrode capacitance, which can be detected by impedance measurements or other type of AC measurements. Accordingly, the activity monitoring unit 105′ of the embodiment illustrated in FIG. 3 further comprises impedance determination circuitry including an AC current source 112 and a voltmeter 113.

In another embodiment, the pH sensor is used for long-term monitoring of blood pH. Changes in blood pH may occur due to medication, and monitoring the pH may help the physician to titrate the proper dosage of medication. pH also may change due to sleep apnea or other metabolic disease states that cause alkalosis or acidosis. The monitor would allow for a physician to monitor the pH trend of the patient and prescribe long-term treatment accordingly.

In yet another embodiment, the mechanical motion of the heart chamber is sensed by measuring the potential of the dissimilar electrode materials in response to contact with the inner heart wall. When the heart wall contacts the electrodes, the thin layer of electrons is perturbed, thereby changing the measured potential. The force of the cardiac contraction will alter the potential change on the electrodes. Measurement of the cardiac contraction would occur during systole. Therefore, constant monitoring of the electrode potential during systole and diastole allows for cardiac contractile force measurement as well as venous pH measurements.

For pH sensors, it is preferred to identify materials that undergo a reversible reduction-oxidation reaction when in solution, such that the ratio of activation energies maximizes the resulting cell potential as predicted by the Nernst equation. In the case of iridium oxide, this ratio is facilitated by the transition of Ir from a 4+ to a 3+ charge state (in the form of IrO2 and Ir2O3, respectively). Since the ratio of activation energies is dependent on the molar concentrations of the ions in solutions (and therefore driven by pH), one can see that the voltage sensitivity of the redox reaction can be changed by altering the amount of molar concentration of highly active constituents. In this regard, a preferred iridium oxide coating may actually be a non-stoichiometric composition, meaning that the deposited coating is either IrO(2−x) or IrO(2+x). In this regard, it is possible to tailor the Nernst potential in such a way that it matches specific device and/or sensor requirements.

In a preferred embodiment, the IrOx is deposited on the surface of the device or device component using reactive physical vapor deposition (PVD), although chemical vapor deposition (CVD), or related techniques (PE-CVD, ion beam, etc.) may be used. In reactive PVD, the stoichiometry of the resulting IrOx coating is dependent on the amount of oxygen gas injected into the carrier gas (typically Ar), and the amount of iridium sputtered from a pure target. In this manner, it is possible to create a variety of iridium oxide compositions; the most preferred stoichiometry would be IrO(1.5).

In a preferred embodiment, an IrOx coating is developed such that it exhibits Nernstian or super-Nernstian behavior, and is paired with a counter electrode that provides sub-Nernstian behavior. It is possible that this counter electrode could be a different IrOx composition, as well as PEDOT, bare metal, or other materials.

Although an exemplary embodiment of the present invention has been shown and described, it should be apparent to those of ordinary skill that a number of changes and modifications to the invention may be made without departing from the spirit and scope of the present invention. In particular, it is possible to integrate the activity monitor according to the present invention in devices other than pacemakers. This invention can readily be adapted to a number of different kinds of medical devices by following the present teachings. All such changes, modifications and alterations should therefore be recognized as falling within the scope of the present invention.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

Additionally, the disclosure of a range of values is a disclosure of every numerical value within that range.

### LIST OF REFERENCE NUMERALS

100 (elongate) housing

101, 102 electrodes

104 amplifier

105, 105′ activity monitoring unit

106 pacing control unit

107 stimulation unit

108 sensing unit

109 memory

110 communication unit

111 iridium oxide electrode

112 AC current source

113 voltmeter

I/we claim:

1. An activity monitor for monitoring metabolic demand comprising:
at least two electrodes that are made of biocompatible materials that have different sensitivities to pH changes, said at least two electrodes being connected to an activity monitoring unit that is adapted to determine an open-circuit potential difference between said at least two electrodes and to generate an activity signal derived from said open-circuit potential difference.

2. The activity monitor of claim 1, wherein one of said at least two electrodes is made of or comprises a first biocompatible material that presents a Nernstian or super-Nernstian behavior with pH, and the other of said at least two electrode is made of or comprises a second biocompatible material that is either in-sensitive to pH or presents a sub-Nernstian behavior.

3. The activity monitor of claim 2, wherein said first biocompatible material that presents a Nernstian or super-Nernstian behavior is a material exhibiting a variation of open-circuit potential with a large negative slope of less than −50 mV/pH, preferably less than −60 mV/pH, and wherein said second biocompatible material, that presents a sub-Nernstian behavior, is a material exhibiting a variation of open-circuit potential with a smaller negative slope of more than −40 mV/pH, preferably more than −30 mV/pH.

4. The activity monitor of claim 2, wherein said first biocompatible material that presents a Nernstian or super-Nernstian behavior is a material exhibiting a variation of open-circuit potential with a large negative slope of less than −60 mV/pH, and wherein said second biocompatible material, that presents a sub-Nernstian behavior, is a material exhibiting a variation of open-circuit potential with a smaller negative slope of more than −30 mV/pH.

5. The activity monitor according to claim 2, wherein the sub-Nernstian material is a coating made of or comprising poly(3,4-ethylenedioxythiophene):poly(styrenesulfonate).

6. The activity monitor according to at least one of claim 2, wherein the super-Nernstian material is a coating made of or comprising iridium oxide.

7. The activity monitor according to claim 6, wherein the super-Nernstian material is a coating made of or comprising a non-stoichiometric composition of iridium oxide comprising either IrO(2−x) or IrO(2+x).

8. The activity monitor according to claim 7, wherein the super-Nernstian material is a coating made of or comprising IrO(1.5) as a non-stoichiometric composition of iridium oxide.

9. The activity monitor according to at least one of claim 6, wherein the coating made of or comprising iridium oxide is deposited on the electrode surface via reactive physical vapor deposition (PVD).

10. An implantable leadless pacemaker comprising an activity monitor for monitoring metabolic demand according to claim 1, said implantable leadless pacemaker having a housing, said two electrodes being arranged on said housing, wherein said activity monitoring unit is adapted to periodically determine the open-circuit potential difference between said two electrodes, said implantable leadless pacemaker further comprising a control unit that is connected to said activity monitoring unit and that is adapted to adjust a pacing rate in response to said activity signal generated by said activity monitoring unit.

11. The implantable leadless pacemaker according to claim 10, wherein the activity monitoring unit or the control unit, or both, are configured to generate an activity signal indicating an increase of pacing rate or an increasing pacing rate, respectively, only when a sudden decrease of pH is determined.

12. The implantable leadless pacemaker according to claim 10, wherein the activity monitoring unit or the control unit, or both, are configured to determine or to respond to the activity signal during diastole only.

13. The implantable leadless pacemaker according to claim 10, wherein the activity monitoring unit or the control unit or both are configured evaluate the activity signal so as to determine a contact with an inner heart wall and thus to monitor cardiac contraction during systole.

14. The implantable leadless pacemaker according to claim 10, further comprising:
an impedance determination circuitry; and
a third electrode, said third electrode is made of or comprising iridium oxide and is connected to said impedance determination circuitry, wherein said impedance determination circuitry is configured to determine a tissue-electrode capacitance or changes thereof.

15. The implantable leadless pacemaker according to claim 14, wherein said third electrode has a smaller surface than said two electrodes.

16. The activity monitor for monitoring metabolic demand according to claim 1, further being configured perform long-term monitoring of pH.

17. The implantable leadless pacemaker according claim 10, further being configured perform long-term monitoring of pH.

18. An implantable leadless pacemaker comprising:
a housing;
at least two electrodes arranged on said housing, wherein said two electrodes are made of biocompatible materials that have different sensitivities to pH changes;
an activity monitoring unit arranged within the housing and connected to said two electrodes, said activity monitoring unit configured to periodically determine an open-circuit potential difference between said two electrodes and to generate an activity signal derived from said open-circuit potential difference; and
a control unit arranged within the housing and connected to said activity monitoring unit, said control unit configured to adjust a pacing rate in response to said activity signal generated by said activity monitoring unit.
